(19) United States
(12) Patent Application Publication
(10) Pub. No.: US 2011/0052516 A1
(43) Pub. Date: Mar. 3, 2011

(54) SUNSCREENS

(76) Inventor: Christine Mendrok-Edinger, Rheinfelden-Minseln (DE)

(21) Appl. No.: 12/991,300

(22) PCT Filed: May 15, 2009
(86) PCT No.: PCT/EP2009/055902

§ 371 Date: Nov. 5, 2010

(30) Foreign Application Priority Data
May 16, 2008 (EP) 08009080.6

(51) Int. Cl. A61K 8/55; A61Q 17/04; A61K 8/58

(52) U.S. Cl. 424/60; 424/59

(57) ABSTRACT

The present invention relates to the use of phosphate ester surfactants for increasing the sun protection factors (SPF's) of topical compositions which comprise at least on UV-filter substance. Furthermore, the invention relates to topical compositions comprising a phosphate ester surfactant and at least 4 wt-% of butyl methoxydibenzoylmethane in combination with further UV-filter substances.

The present invention relates to the use of phosphate ester surfactants for increasing the sun protection factors (SPF's) of topical compositions which comprise at least one UV-filter substance. Furthermore, the invention relates to topical compositions comprising a phosphate ester surfactant and at least 4 wt-% of butyl methoxydibenzoylmethane in combination with further UV-filter substances.

Sun care products have evolved considerably over the years. Earlier formulations were intended to protect the user from UV-B radiation (UVB) as was once thought that UV-B rays were the most important contributors to wrinkling, skin disease, and skin cancer. However, more recent studies have shown that UV-A radiation (UVA) is equally or even more important in the development of solar damage and skin diseases, such as lupus erythematosus and melanoma and non-melanoma skin cancers. Thus, today's focus is toward eliminating as much of UVA (320-400 nm) and/or UVB (280-320 nm) light as possible. Consequently, there's a constantly increasing need for sun care products exhibiting high SPF's up to 50+ and high UVA protection.

The SPF (Sun Protection Factor) rating system has been developed to provide consumer guidance in selecting sun care products. SPF is measured in the laboratory with a solar simulator that induces UV erythema. However, erythema is primarily caused by UVB, making SPF testing primarily a measurement of UVB—not UVA—protection.

Colipa has developed a method for testing the UVA performance of sun care products that meet the European Commission's Recommendation of 22 Sep. 2006 on the efficacy of sunscreen products. The UVA protection performance according to the invention is determined according to the ‘Method for the in vitro determination of UVA protection provided by sunscreen products’ (COLIPA Guideline 2007) which is labeled as UVAPF (UVA protection factor).

Many UV-filter substances have been developed in the past protecting against the harmful effect of UVA and/or UV-B radiation and even shorter radiation (UVC). These substances are usually incorporated either alone or in combination with each other into cosmetic or pharmaceutical preparations which are widely known and used.

Due to the increasing demand for high SPF sun care products with a UVA protection complying with the above mentioned standards, more UV-filter substances at elevated levels have to be incorporated into the sun care products; this, however, is not always feasible, as high UV-filter substance concentrations add considerable cost to the formulation and often lead to an unpleasant skin feel and/or aesthetic appearance.

Furthermore, high sunscreen levels can promote increased irritancy. In order to obtain a UVA protection complying with the above mentioned standards high concentrations of at least one UVA filter substance has to be incorporated into the sun care products. However, not many UVA-filter substances are commercially available and all of them exhibit more or less severe downsides e.g. in view of stability, solubility and/or suitable absorption profile in order to achieve the recommended UVA protection at a reasonable concentration level. For example, the frequently used butyl methoxydibenzoylmethane is difficult to be formulated in high amounts into sun care products as it often renders the formulation instable and as it is incompatible with many other UV-filter substances. Thus, there is an ongoing need for ingredients which improve the functionality of UV-filter substances in order to achieve higher SPF at a given concentration of UV-filter substances and furthermore allow the incorporation of high amounts of butyl methoxydibenzoylmethane in order to achieve the recommended UVA protection.

Surprisingly, it has been found that phosphate ester surfactants are able to enhance the in vivo SPF at given UV-filter substance concentrations. This surprising effect helps to overcome the problems of the prior art outlined above. Furthermore it has been found that the incorporation of high levels of butyl methoxydibenzoylmethane is facilitated which makes it possible to achieve the recommended UVA protection at high SPF levels.

The in vivo SPF according to the invention is tested according to the Colipa, International Test Method for the in vivo determination of the Sun Protection Factor SPF provided by sunscreen products, guideline 2006.

Thus, the invention relates in one aspect to the use of a phosphate ester surfactant for increasing the SPF of a topical composition comprising at least one UV-filter substance. Preferably, the at least one UV-filter substance is a UVB and/or broadband-filter substance.

In another aspect the invention relates to the use of a phosphate ester surfactant for increasing the SPF of a topical composition comprising butyl methoxydibenzoylmethane and at least one UV-filter substance selected from a UVB and/or broadband-filter substance.

Phosphate esters surfactants suitable for incorporation into the compositions of the present invention have the formula

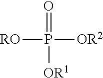

The phosphate ester surfactant has the general structure wherein R, R¹ and R² may be hydrogen, an alkyl of from 1 to about 22 carbons, preferably from about 12 to 18 carbons, or an alkoxylated alkyl of from 1 to about 22 carbons, preferably from about 12 to 18 carbons, and having 1 or more, preferably from about 2 to about 25, most preferably 2 to 12, moles ethylene oxide, with the proviso that at least one of R, R¹ and R² is an alkyl or alkoxylated alkyl as previously defined but having at least 6 alkyl carbons in said alkyl or alkoxylated alkyl group.

Monoesters in which R¹ and R² are hydrogen and R is selected from alkyls of 10 to 18 carbons and alkoxylated fatty alcohols of 10 to 18 carbons and 2 to 12 moles ethylene oxide are preferred. Among the preferred phosphate ester surfactants, mention may be made of, C8-10 Alkyl Ethyl Phosphate, C9-15 Alkyl Phosphate, Ceteareth-2 Phosphate, Ceteareth-5 Phosphate, Ceteth-8 Phosphate, Ceteth-10 Phosphate, Cetyl Phosphate, C6-10 Pareth-4 Phosphate, C12-15 Pareth-2 Phosphate, C12-15 Pareth-3 Phosphate, DEA-Ceteareth-2 Phosphate, DEA-Cetyl Phosphate, DEA-Oleth-3 Phosphate, Potassium cetyl phosphate, Deceth-4 Phosphate and Deceth-6 Phosphate. Particular preferred phosphate ester surfactants according to the invention are cetyl phosphate, potassium cetyl phosphate and/or DEA cetyl phosphate.

The phosphate ester surfactant is generally present in the topical compositions according to the invention in proportions ranging from 0.1 to 5 wt.-%, preferably from 0.5 to 5 wt.-%, most preferably from 1 to 3 wt.-%, such as particularly from 2 to 3 wt.-% with respect to the total weight of the topical composition.

The at least one UV-filter substance is preferably selected from conventional UVA and/or UVB and/or broad spectrum UV-filter substances known to be added into topical compositions such as cosmetic or dermatological sun care products. Such UV-filter substances comprise all groups which absorb light in the range of wavelengths 400 nm to 320 nm (UVA) and 320 nm to 280 nm (UVB) or of even shorter wavelengths (UVC) and which are or can be used as cosmetically acceptable UV-filter substances. Such UV-filter substances are e.g. listed in the CTFA Cosmetic ingredient Handbook or “The Encyclopedia of Ultraviolet Filters” (ISBN: 978-1-932633-25-2) by Nadim A. Shaath.

Suitable UV-filter substances may be organic or inorganic compounds. Exemplary organic UV-filter substances encompass e.g. acrylates such as e.g. 2-ethylhexyl 2-cyano-3,3-diphenylacrylate (octocrylene, PARSOL® 340), ethyl 2-cyano-3,3-diphenylacrylate;

Camphor derivatives such as e.g. 4-methyl benzylidene camphor (PARSOL® 5000), 3-benzylidene camphor, camphor benzalkonium methosulfate, polyacrylamidomethyl benzylidene camphor, sulfo benzylidene camphor, sulphomethyl benzylidene camphor, terephthalylidene dicamphor sulfonic acid (Mexoryl® SX); Cinnamate derivatives such as e.g. ethylhexyl methoxycinnamate (PARSOL® MCX), ethoxyethyl methoxycinnamate, isoamyl methoxycinnamate as well as cinnamic acid derivatives bond to siloxanes;

p-Aminobenzoic acid derivatives such as e.g. p-aminobenzoic acid, 2-ethylhexyl p-dimethylaminobenzoate, N-oxypropylenated ethyl p-aminobenzoate, glyceryl p-aminobenzoate; Benzophenones such as e.g. benzophenone-3, benzophenone-4,2,2′,4,4′-tetrahydroxy-benzophenone, 2,2′-dihydroxy-4,4′-dimethoxybenzophenone; Esters of benzalmalonic acid such as e.g. di-(2-ethylhexyl) 4-methoxybenzalmalonate;

Organosiloxane compounds carrying chromophore groups such as e.g. polysilicones-15 (PARSOL® SLX), drometrizole trisiloxane (Mexoryl® XL); Imidazole derivatives such as e.g. 2-phenyl benzimidazole sulfonic acid (PARSOL®HS) and salts thereof such as e.g. sodium- or potassium salts, ammonium salts, morpholine salts, salts of primary, sec. and tert. amines like monoethanolamine salts, diethanolamine salts; Salicylate derivatives such as e.g. isopropylbenzyl salicylate, benzyl salicylate, butyl salicylate, ethylhexyl salicylate (PARSOL® EHS, Neo Heliopan® OS), isooctyl salicylate or homomethyl salicylate (homosalate, PARSOL® HMS, Neo Heliopan® HMS); Triazine derivatives such as e.g. ethylhexyl triazone (Uvinul® T-150), diethylhexyl butamido triazone (Uvasorb® HEB), bis-ethylhexyloxyphenol methoxyphenyl triazine (Tinosorb® S); Benzotriazole derivatives such as e.g. 2,2′-methylene-bis-(6-(2H-benzotriazole-2-yl)-4-(1,1,3,3-tetramethylbutyl)-phenol (Tinosorb® M); Encapsulated UV-filters such as e.g. encapsulated ethylhexyl methoxycinnamate (Eusolex® UV-pearls) or microcapsules loaded with UV-filters as e.g. disclosed in EP 1471995; Dibenzoylmethane derivatives such as e.g. 4-tert.-butyl-4′-methoxydibenzoyl-methane (PARSOL® 1789), dimethoxydibenzoylmethane, isopropyldibenzoylmethane; Phenylene-1,4-bis-benzimidazolsulfonic acids or salts such as e.g. 2,2-(1,4-phenylene)bis-(1H-benzimidazol-4,6-disulfonic acid) (Neoheliopan AP); Amino substituted hydroxybenzophenones such as e.g. 2-(4-diethylamino-2-hydroxy-benzoyl)-benzoic acid hexylester (Aminobenzophenon, Uvinul® A Plus); Benzoxazol-derivatives such as e.g. 2,4-bis-[5-1(dimethylpropyl)benzoxazol-2-yl-(4-phenyl)-imino]-6-(2-ethylhexyl)-imino-1,3,5-triazin [Uvasorb® K2A);

Inorganic UV-filter substances encompass pigments such as e.g. microparticulated Zinc oxide or Titanium dioxide (e.g. commercially available as PARSOL® TX) The term “microparticulated” refers to a particle size from about 5 nm to about 200 nm, particularly from about 15 nm to about 100 nm. The particles may also be coated by other metal oxides such as e.g. aluminum or zirconium oxides or by organic coatings such as e.g. polyols, methicone, aluminum stearate, alkyl silane. Such coatings are well known in the art.

In order to enhance the photostability of sun care products it may be desirable to add a photostabilizer. Exemplary photostabilizers known to a skilled person in the art encompass e.g. 3,3-diphenylacrylate derivatives such as e.g. octocrylene (PARSOL® 340) or Polyester-8 (Polycrylene®); Benzylidene camphor derivatives such as e.g. 4-methyl benzylidene camphor (PARSOL® 5000); Benzalmalonate derivatives such as e.g. polysilicones-15 (PARSOL® SLX) or diethylhexyl syringylidene malonate (Oxynex ST liquid); Dialkyl naphthalates such as diethylhexyl naphthalate (Corapan TQ) without being limited thereto. An overview on further stabilizers is e.g. given in ‘SPF Boosters & Photostability of Ultraviolet Filters’, HAPPI, October 2007, p. 77-83 which is included herein by reference. The photostabilizers are generally used in an amount of 0.05 to 10 wt.-% with respect to the total weigh of the topical composition.

Generally, the amount of each UV-filter substance in the topical compositions according to the invention is selected in the range of about 0.1 to 10 wt.-%, preferably in the range of about 0.2 to 7 wt.-%, most preferably in the range of about 0.5 to 5 wt.-% with respect to the total weigh of the topical composition.

The total amount of UVA-filter substance(s), in particular of butyl methoxydibenzoylmethane, in the topical compositions according to the invention is preferable selected in the range of about 2 to 8 wt.-%, in particular in the range of about 4 to 6 wt.-%, most particular in the range of about 3 to 5 wt.-%, such as 4 to 5 wt.-% with respect to the total weight of the topical composition.

The total amount of UV-filter substances in the topical compositions according to the invention is preferably in the range of about 1 to 40 wt.-%, preferably in the range of about 5 to 30 wt.-%, in particular in the range of 10 to 30 wt.-%, such as 20 to 30 wt.-% with respect to the total weight of the topical composition.

Preferred UVB-filter substances according to the invention encompass polysilicones-15, phenylbenzimidazol sulfonic acid, octocrylene, ethylhexyl methoxycinnamate, ethyl hexylsalicylate and/or homosalate.

Preferred broadband UV-filter substances according to the invention encompass unsymmetrical s-triazine derivatives such 2,4-Bis-{[4-(2-ethyl-hexyloxy)-2-hydroxy]-phenyl}-6-(4-methoxyphenyl)-1,3,5-triazin, certain benzophenones such as e.g. 2-Hydroxy-4-methoxy-benzophenon, 2,2′-Methylen-bis-(6-(2H-benzotriazol-2-yl)-4-(1,1,3,3-tetramethyl-butyl)-phenol), and/or titanium dioxide.

The preferred UVA-filter substance according to the invention is butyl methoxydibenzoylmethane. Preferably, butyl methoxydibenzoylmethane is the only UVA-filter substance in the topical compositions according to the invention.

In a particular embodiment the invention relates to the use of a phosphate ester surfactant selected from cetyl phosphate, potassium cetyl phosphate and/or DEA cetyl phosphate for increasing the SPF of a topical composition comprising butyl methoxydibenzoylmethane and at least one UV-filter substance selected from a UVB and/or broadband-filter substance. Preferably, the topical composition comprises butyl methoxydibenzoylmethane, polysilicones-15 and phenylbenzimidazol sulfonic acid and at least one further UV-B and/or broadband filter substances selected from octocrylene, titanium dioxide, ethyl hexylsalicylate and/or homosalate. In a most preferred embodiment, the topical composition comprises at least 4.5 wt.-% butyl methoxydibenzoylmethane, at least 2 wt.-% of polysilicones-15 and at least 2 wt.-% of phenylbenzimidazol sulfonic acid, at least 3.6 wt.-% of octocrylene and at least one further UV-B and/or broadband filter substances selected from titanium dioxide, ethyl hexylsalicylate and/or homosalate.

Surprisingly, it has furthermore been found that photostable, high SPF compositions which comply with the above mentioned UVA standards can be achieved by combining at least 4.5 wt.-% of butyl methoxydibenzoylmethane with at least 2 wt.-% of polysilicones-15 and at least 2 wt.-% phenylbenzimidazol sulfonic in combination with at least one further UVB or broadband filter substance which preferably is selected from octocrylene, titanium dioxide, ethyl hexylsalicylate and/or homosalate in the presence of a phosphate ester surfactant.

Thus, in another aspect the invention relates to a topical composition comprising
a phosphate ester surfactant,
at least 4.5 wt.-% of butyl methoxydibenzoylmethane, preferably about 4.5 to 6 wt.-%, in particular about 5 wt.-%,
at least 2 wt.-% of polysilicone-15, preferably about 2 to 10 wt.-%, in particular about 3 wt.-%,
at least 2 wt.-% of phenylbenzimidazol sulfonic, preferably about 2 to 8 wt.-%,
preferably about 3 to 4 wt.-%, in particular about 4 wt.-% in a cosmetically acceptable carrier.
and wherein the topical composition further comprises at least one further UVB and/or broadband filter substance which preferably is selected from octocrylene, titanium dioxide, ethyl hexylsalicylate and/or homosalate. Preferably, the phosphate ester surfactant is cetyl phosphate, potassium cetyl phosphate and/or DEA cetyl phosphate and is used in a concentration of about 1 to 3 wt.-%, such as particularly 2 to 3 wt.-% with respect to the total weigh of the topical composition. Preferably, the topical composition further contains at least 3.6 wt.-% of octocrylene.

Preferably, the total amount of the at least one further UVB and/or broadband filter substance is at least 8 wt.-%, preferably in the range of about 8 to 20 wt.-% and the total amount of UV-filter substances is in the range of about 20 to 30 wt.-% with respect to the total weight of the topical composition.

Particular preferred are topical compositions as outlined above comprising
2 to 3 wt.-% of cetyl phosphate, potassium cetyl phosphate and/or DEA cetyl phosphate,
4.5 to 5 wt.-% of butyl methoxydibenzoylmethane,
2 to 3 wt.-% of polysilicone-15,
3 to 4 wt.-% of phenylbenzimidazol sulfonic and
3.6 to 10 wt.-% of octocrylene in a cosmetically acceptable carrier
and wherein the topical composition further comprises at least one further UV-filter substance selected from titanium dioxide, ethyl hexylsalicylate and/or homosalate.

Further preferred are topical compositions as outlined above comprising
2 to 3 wt.-% of cetyl phosphate, potassium cetyl phosphate and/or DEA cetyl phosphate,
5 wt.-% of butyl methoxydibenzoylmethane,
3 wt.-% of polysilicone-15,
4 wt.-% of phenylbenzimidazol sulfonic and
3.6 to 10 wt.-% of octocrylene in a cosmetically acceptable carrier
and wherein the topical composition further comprises at least one further UV-filter substance selected from titanium dioxide, ethyl hexylsalicylate and/or homosalate.

Unexpectedly, it has furthermore been found that photostable, high SPF compositions which comply with the above mentioned UVA standards can also be achieved by combining at least 4 wt.-% of butyl methoxydibenzoylmethane with at least 2 wt.-% of bis-ethylhexyloxyphenol methoxyphenol triazine and at least 2 wt.-% phenylbenzimidazol sulfonic in combination with at least one further UVB or broadband filter substance which preferably is selected from octocrylene, titanium dioxide, ethyl hexylsalicylate and/or homosalate in the presence of a phosphate ester surfactant.

In another embodiment the invention relates to a topical composition comprising
a phosphate ester surfactant,
at least 4 wt.-% of butyl methoxydibenzoylmethane, preferably about 4 to 6 wt.-%, in particular about 4 wt.-%,
at least 2 wt.-% of bis-ethylhexyloxyphenol methoxyphenol triazine, preferably about 2 to 5 wt.-%, in particular about 3 wt.-%,
at least 2 wt.-% of phenylbenzimidazol sulfonic, preferably about 2 to 8 wt.-%, in particular about 4 wt.-% in a cosmetically acceptable carrier
and wherein the topical composition further comprises at least one further UVB and/or broadband filter substance which preferably is selected from octocrylene, titanium dioxide, ethyl hexylsalicylate, ethylhexylmethoxycinnamate and/or homosalate a cosmetically acceptable carrier. Preferably, the phosphate ester surfactant is cetyl phosphate, potassium cetyl phosphate and/or DEA cetyl phosphate and is used in a concentration of about 2 to 3 wt.-% with respect to the total weigh of the topical composition. Preferably, the topical composition further contains at least 3.6 wt.-% of octocrylene.

Preferably, the total amount of the at least one further UVB and/or broadband filter substance is at least 8 wt.-%, preferably in the range of about 8 to 20 wt.-% and the total amount of UV-filter substances is in the range of about 20 to 30 wt.-% with respect to the total weight of the topical composition.

Particular preferred are topical compositions as outlined above comprising
2 to 3 wt.-% of cetyl phosphate, potassium cetyl phosphate and/or DEA cetyl phosphate,
3 to 5 wt.-% of butyl methoxydibenzoylmethane
2 to 3 wt.-% of bis-ethylhexyloxyphenol methoxyphenol triazine,
3 to 4 wt.-% of phenylbenzimidazol sulfonic and
3.6 to 10 wt.-% of octocrylene in a cosmetically acceptable carrier
and wherein the topical composition further comprises at least one further UV-filter substance selected from titanium dioxide, ethyl hexylsalicylate and/or homosalate.

Further preferred are topical compositions as outlined above comprising
2 to 3 wt.-% of cetyl phosphate, potassium cetyl phosphate and/or DEA cetyl phosphate,
4 wt.-% of butyl methoxydibenzoylmethane
3 wt.-% of bis-ethylhexyloxyphenol methoxyphenol triazine,
4 wt.-% of phenylbenzimidazol sulfonic and
3.6 to 10 wt.-% of octocrylene in a cosmetically acceptable carrier
and wherein the topical composition further at least one further UV-filter substance selected from titanium dioxide, ethyl hexylsalicylate and/or homosalate.

In a particular embodiment all topical compositions according to the invention further comprise a co-surfactant in an amount of 1 to 4 wt.-%, in particular about 2 wt.-% in order to further improve the stability of the formulation. Preferably, the co-surfactant is selected from Lanette 0 (Cetearyl Alcohol (mixture of C16 and C18 alcohol)), Lanette 16/Lorol C 16 (Cetyl Alcohol) and/or Estol 3650 (Glyceryl Myristate).

The term “topical composition” as used herein refers in particular to cosmetic compositions that can be topically applied to mammalian keratinous tissue such as e.g. human skin or hair, particularly human skin.

The term “cosmetic composition” as used in the present application refers to cosmetic compositions as defined under the heading “Kosmetika” in Römpp Lexikon Chemie, 10th edition 1997, Georg Thieme Verlag Stuttgart, New York as well as to cosmetic preparations as disclosed in A. Domsch, “Cosmetic Preparations”, Verlag für chemische Industrie (ed. H. Ziolkowsky), 4^{th} edition, 1992.

The term cosmetically acceptable carrier refers to all carriers and/or excipients and/or diluents conventionally used in topical preparations

Preferably, the topical preparations according to the present invention are in the form of a suspension or dispersion in solvents or fatty substances, or alternatively in the form of an emulsion or micro emulsion (in particular of O/W-type), PIT-emulsion, multiple emulsion (e.g. O/W/O-type and W/O/W), pickering emulsion, one- or multiphase solution or vesicular dispersion or other usual forms, which can also be applied by pens, as masks or as sprays. If the topical preparation is or comprises an emulsion it can also contain one or more anionic, nonionic, cationic or amphoteric surfactant(s).

In a particular preferred embodiment, the topical compositions according to the invention are O/W emulsions.

The oil phase of the O/W emulsions according to the invention preferably comprises oils selected from butylenglykoldicaprylat/-dicaprat, dicaprylylether, C₁₂₋₁₅-Alkylbenzoat, C₁₈₋₃₈-fatty acid triglyceride, dibutyladipate, cyclomethicone, 2-phenylethylbenzoat, isopropyl lauroyl sarkosinate, diisopropyl sebacate as well as mixtures thereof. The amount of the oil (one or several) in the O/W emulsion according to the invention is generally selected in the range of about 0.1 bis 40 wt.-%, in particular in the range of about 1.0 to 30 wt.-%, most in particular in the range of about 5% to 20 wt.-% with respect to the total weight of the topical composition.

Preferred topical compositions according to the invention are sun care preparations.

Topical compositions in accordance with the invention can be in the form of a liquid, lotion, a thickened lotion, a gel, a cream, a milk, an ointment, a paste or a make-up, and can be optionally be packaged as an aerosol and can be provided in the form of a mousse such as a aerosol mousse, a foam or a spray foam, a spray, a stick.

In accordance with the present invention, the topical compositions according to the invention may optionally be combined with further ingredients such as ingredients for skin lightening; tanning prevention; treatment of hyperpigmentation; preventing or reducing acne, wrinkles, lines, atrophy and/or inflammation; chelators and/or sequestrants; anti-cellulites and slimming (e.g. phytanic acid), firming, moisturizing and energizing, self tanning, soothing, as well as agents to improve elasticity and skin barrier and/or further UV-filter substances and carriers and/or excipients or diluents conventionally used in topical compositions. If nothing else is stated, the excipients, additives, diluents, etc. mentioned in the following are suitable for topical preparations according to the present invention. The necessary amounts of the cosmetic and dermatological adjuvants and additives can, based on the desired product, easily be determined by the skilled person.

The cosmetically active ingredients useful herein can in some instances provide more than one benefit or operate via more than one mode of action.

Preferred examples of further ingredients are vitamin C (ascorbic acid) and/or its derivatives (e.g. ascorbyl phosphate such as Stay C (sodium ascorbyl monophosphate) from DSM Nutritional Products Ltd.), vitamin A and/or its derivatives (e.g., retinoid derivatives such as retinyl palmitate or retinyl propionate), vitamin E and/or its derivatives (e.g., tocopherol acetate), vitamin B₆, vitamin B₁₂, biotin, co-enzyme Q10, EGCG, hydroxytyrosol and/or olive extract, shea butter, algae extract, cocoa butter, aloe extract, jojoba oil, echinacea extract, chamomile extract, Glycyrrhetinic Acid, Glycyryca Glabra extract, in particular vitamin E and/or its derivatives, shea butter, algae extract, cocoa butter, aloe extract and/or vitamin A and/or its derivatives. The additional cosmetically active ingredient is typically included in an amount of at least 0.001 wt. % based on the total weight of the topical preparation. Generally, an amount of about 0.001 wt. % to about 30 wt. %, preferably from about 0.001 wt. % to about 10 wt. % of an additional cosmetically active agent is used.

A vitamin E derivative for use in the present invention is tocopheryl acetate. Tocopheryl acetate may be present in the topical preparations in an amount from about 0.05 wt.-% to about 25 wt.-%, in particular 0.5 wt.-% to 5 wt.-%. Another vitamin E derivative of interest is tocopheryl linoleate. Tocopheryl linoleate may be present in the skin care composition in an amount from about 0.05 wt.-% to about 25 wt.-% in particular 0.05 wt.-% to 5 wt.-%.

Vitamin A and/or its derivatives in particular retinoid derivatives such as retinyl palmitate or retinyl propionate is preferably used in the topical preparations according to the invention in an amount of 0.01-5 wt.-%, in particular 0.01-0.3 wt.-%

The topical cosmetic compositions of the invention can also contain usual cosmetic adjuvants and additives, such as preservatives/antioxidants, fatty substances/oils, water, organic solvents, silicones, thickeners, softeners, emulsifiers, sunscreens, antifoaming agents, moisturizers, aesthetic components such as fragrances, surfactants, fillers, sequestering agents, anionic, cationic, nonionic or amphoteric polymers or mixtures thereof, propellants, acidifying or basifying agents, dyes, colorings/colorants, abrasives, absorbents, essential oils, skin sensates, astringents, antifoaming agents, pigments or nanopigments, e.g. those suited for providing a photoprotective effect by physically blocking out ultraviolet radiation, or any other ingredients usually formulated into cosmetic compositions. Such cosmetic ingredients commonly used in the skin care industry, which are suitable for use in the compositions of the present invention are e.g. described in the CTFA Cosmetic Ingredient Handbook, Second Edition (1992) without being limited thereto.

The necessary amounts of the cosmetic and dermatological adjuvants and additives can—based on the desired product—easily be chosen by a skilled person in this field and will be illustrated in the examples, without being limited hereto.

Of course, one skilled in this art will take care to select the above mentioned optional additional compound or compounds and/or their amounts such that the advantageous properties intrinsically associated with the combination in accordance with the invention are not, or not substantially, detrimentally affected by the envisaged addition or additions.

The cosmetic and/or dermatological compositions according to the invention have a pH in the range of 3-10, preferably in the range of pH of 4-8, most preferred in the range of pH 6-8.

The following examples are provided to further illustrate the compositions and effects of the present invention. These examples are illustrative only and are not intended to limit the scope of the invention in any way.

### EXAMPLE 1

The same UV-filter substances were incorporated in two different standard cosmetic bases using Potassium Cetyl Phosphate, respectively Polyester-5 & Water & Butylene Glycol & Glycerin as emulsifier. The in vivo SPF was tested according to the Colipa, International Test Method for the in vivo determination of the Sun Protection Factor SPF provided by sunscreen products, guideline 2006.

TABLE 1

| Phase | INCI | wt.-% | wt.-% |
|---|---|---|---|
| A | Polysilicone-15 (Parsol ® SLX) | 3 | 3 |
| | Butyl Methoxydibenzoylmethane | 4 | 4 |
| | (Parsol ® 1789) | | |
| | Phenylbenzimidazole Sulfonic Acid | 4 | 4 |
| | (Parsol ® HS) | | |
| | Potassium Cetyl Phosphate (Amphisol ® K) | 3 | |
| | Polyester-5 & Water & Butylene Glycol & | | 10 |
| | Glycerin (Lipo PE Base BG-55) | | |
| | in vivo SPF | 36 | 23 |

As can bee seen from table 1 the incorporation of a defined amount of UV-filter substances in a standard potassium cetyl phosphate formulation resulted in a significantly higher in vivo SPF compared to the same UV-filter combination in a standard Polyester-5 & Water & Butylene Glycol & Glycerin formulation.

### EXAMPLE 2

Various UV-filter combinations have been incorporated into a cosmetic base as outlined in table 2 using Potassium Cetyl Phosphate (Amphisol K) or Glyceryl Stearate & PEG-100 Stearate (Arlacel 165) as emulsifier. The use of Potassium Cetyl Phosphate significantly increased the in vivo SPF of the formulations.

TABLE 2

| | INCI | Wt.-% | | | | | | | | | | | |
|---|---|---|---|---|---|---|---|---|---|---|---|---|---|
| A | Butyl Methoxy- | 5 | 5 | 5 | 5 | 5 | 5 | 4 | 4 | 4 | 4 | 4 | 4 |
| | dibenzoylmethane | | | | | | | | | | | | |
| | Polysilicone-15 | 3 | 3 | 3 | 3 | 3 | 3 | | | | | | |
| | Octocrylene | 8 | 8 | 4.5 | 4.5 | 4.5 | 4.5 | 3.6 | 3.6 | 3.6 | 3.6 | 3.6 | 3.6 |
| | Titanium Dioxide | 6 | 6 | 3 | 3 | | | 3 | 3 | 3 | 3 | 3 | 3 |
| | Bis-Ethylhexyloxy | | | | | | | 3 | 3 | 3 | 3 | 3 | 3 |
| | phenol Methoxy phenol | | | | | | | | | | | | |
| | Triazine | | | | | | | | | | | | |
| | Ethylhexyl Salicylate | | | 5 | 5 | 5 | 5 | | | 5 | 5 | 3 | 3 |
| | Homosalate | | | | | 7.5 | 7.5 | | | | | 5 | 5 |
| | Ethylhexyl | | | | | | | 3 | 3 | 3 | 3 | | |
| | Methoxycinnamate | | | | | | | | | | | | |
| | Potassium Cetyl | | 2 | | 2.5 | | 2 | | 2.5 | | 2 | | 2 |
| | Phosphate | | | | | | | | | | | | |
| | Glyceryl Stearate & | 3.5 | | 3.5 | | 3.5 | | 3.5 | | 3.5 | | 3.5 | |
| | PEG-100 Stearate | | | | | | | | | | | | |
| | Tocopheryl Acetate | 0.5 | 0.5 | 0.5 | 0.5 | 0.5 | 0.5 | 0.5 | 0.5 | 0.5 | 0.5 | 0.5 | 0.5 |
| | Dimethicone 200/100 cs | 0.3 | 0.3 | | 0.3 | 0.3 | 0.3 | | 0.3 | | 0.3 | | 0.3 |
| | Dimethicone 200/350 cs | | | 1 | | | | 1 | | 1 | | 1 | |
| | BHT | 0.05 | | | | | | | | | | | |
| | Fatty alcohol¹⁾ | 1.5 | 2 | 2 | 2 | 2 | 2 | 1.5 | 2 | 2 | 2 | 2 | 2 |
| | Preservative²⁾ | 0.8 | 0.8 | 0.8 | 0.8 | 0.8 | 0.8 | 0.8 | 0.8 | 0.8 | 0.8 | 0.8 | 0.8 |
| | Cyclopentasiloxane & | 1.5 | 1.5 | 2 | 1.5 | 1.5 | 1.5 | 2 | 1.5 | 2 | 1.5 | 2 | 1.5 |
| | Cyclohexasiloxane | | | | | | | | | | | | |
| | Diisopropyl Sebacate | 12 | 12 | 10 | 12 | 10 | 12 | 8 | 12 | 3 | 12 | 5 | 12 |
| | C12-15 Alkyl Benzoate | 12 | 10 | 10 | 10 | 10 | 10 | 15 | 13 | 15 | 10 | 15 | 10 |
| | Butylene Glycol | | | 5 | | 5 | | | | | | 5 | |
| | Dicaprylate/Dicaprate | | | | | | | | | | | | |
| | VP/Eicosene Copolymer | | 2 | | 2 | | 2 | | 2 | | 2 | | 2 |
| B | Triethanolamine | q.s. | | | | | | | | | | | |
| | Glycerin | 3 | 3 | 3 | 3 | 3 | 3 | 3 | 3 | 3 | 3 | 3 | 3 |
| | Xanthan Gum | 0.2 | 0.3 | 0.2 | 0.2 | 0.2 | 0.3 | 0.2 | 0.2 | 0.2 | 0.3 | 0.2 | 0.3 |
| | chelating agent³⁾ | 0.1 | 0.5 | 0.1 | 0.5 | 0.1 | 0.5 | 0.1 | 0.5 | 0.1 | 0.5 | 0.1 | 0.5 |
| | Aqua | Ad 100 | | | | | | | | | | | |
| C | Phenylbenzimidazole | 4 | 4 | 4 | 4 | 4 | 4 | 4 | 4 | 4 | 4 | 4 | 4 |
| | Sulfonic Acid | | | | | | | | | | | | |
| | Aqua | 10 | 10 | 10 | 10 | 10 | 10 | 10 | 10 | 10 | 10 | 10 | 10 |
| | Triethanolamine (TEA) | q.s. | | | | | | | | | | | |
| | in vivo SPF | 46 | 53 | 41 | 76 | 45 | 49 | 45 | 63 | 45 | 59 | 40 | 48 |
| | UVA/UVB = 1/3* | yes | yes | yes | yes | yes | yes | | yes | yes | | yes | |
| | Critical wavelengt | 377 | 377 | 377 | 376 | 376 | | 376 | 375 | 375 | | 376 | |
| | Nr. | 1 | 2 | 3 | 4 | 5 | 6 | 7 | 8 | 9 | 10 | 11 | 12 |
| | Total amount of UV- | 26 | 26 | 24.5 | 24.5 | 29 | 29 | 20.6 | 20.6 | 25.6 | 25.6 | 25.6 | 25.6 |
| | filter substances | | | | | | | | | | | | |

¹⁾selected from Behenyl Alcohol, Cetyl Alcohol or Cetearyl Alcohol
²⁾Phenoxyethanol & Methylparaben & Ethylparaben & Butylparaben & Propylparaben & Isobutylparaben
³⁾selected from Disodium EDTA or Pentasodium Ethylenediamine Tetramethylene Phosphonate.
*EU recommendation fulfilled

Procedure for preparation of the formulations: Heat part A to 85° C. and stir until homogeneous then heat part B to 75° C. and add to part A under agitation, then cool down to 55° C. and add TEA. When everything is homogenous add part C pre-heated to 50° C. Be sure that the pH of Phenylbenzimidazole Sulfonic Acid solution is 7.0 (adjusted with TEA). If traces remain, add small quantities of the used neutralizing base until the particles are dissolved. Homogenize thoroughly and then with continued mixing cool down to ambient temperature. It is generally recommended to use vacuum while producing the emulsion.

As can be seen from table 2, a composition according to the invention with a total amount of UV-filter substances in the range of 20 to 30 wt.-% comprising a phosphate ester surfactant, 5 wt.-% of butyl methoxydibenzoylmethane, 3 wt.-% of polysilicone-15, and 4 wt.-% of phenylbenzimidazol sulfonic acid in combination with further UVB and/or broadband filter substances and realize the criteria of an SPF>50 while achieving the UVA standards, i.e. EU recommendation and critical wavelength whereas the compositions comprising a non phosphate ester surfactant, i.e. Arlacel 165 (A) with the same UV-filter substance concentrations does not achieve the required in vivo SPF (see Formulations 1-4)

As can bee seen from table 2, a composition according to the invention with a total amount of UV-filter substances in the range of 20 to 30 wt.-% comprising a phosphate ester surfactant, 4 wt.-% of butyl methoxydibenzoylmethane, 3 wt.-% of bis-ethylhexyloxyphenol methoxyphenol triazine, and 4 wt.-% of phenylbenzimidazol sulfonic acid in combination with further UVB and/or broadband filter substances realize the criteria of an SPF>50 while achieving the UVA standards, i.e. EU recommendation and critical wavelength whereas the compositions comprising a non phosphate ester surfactant, i.e. Arlacel 165 (A) with the same UV-filter substance concentrations does not achieve the required in vivo SPF (see formulation 7 & 8).

1. A method for increasing the SPF of a topical composition comprising at least one UV-filter substance comprising adding to the composition a phosphate ester surfactant.

2. The method according to claim 1, wherein the topical composition comprises from 0.1 to 5 wt.-% of a phosphate ester surfactant.

3. The method according to claim 1, wherein the topical composition comprises from 1 to 3 wt.-% of a phosphate ester surfactant.

4. The method according to claim 1, wherein the phosphate ester surfactant is selected from cetyl phosphate, potassium cetyl phosphate and/or DEA cetyl phosphate.

5. The method according to claim 1, wherein the topical composition comprises butyl methoxydibenzoylmethane, polysilicone-15 and phenylbenzimidazol sulfonic acid and at least one further UV-B and/or broadband filter substances selected from octocrylene, titanium dioxide, ethyl hexylsalicylate and/or homosalate.

6. The method according to claim 1, wherein the topical composition comprises at least one co-surfactant selected from cetearyl alcohol (mixture of C16 and C18 alcohol), Cetyl Alcohol and/or Glyceryl Myristate.

7. Topical composition comprising
a phosphate ester surfactant,
at least 4.5 wt.-% of butyl methoxydibenzoylmethane
at least 2 wt.-% of polysilicone-15,
at least 2 wt.-% of phenylbenzimidazol sulfonic in a cosmetically acceptable carrier and wherein the topical composition further comprises at least one further UVB and/or broadband filter substance.

8. Topical composition comprising
a phosphate ester surfactant,
at least 4 wt.-% of butyl methoxydibenzoylmethane,
at least 2 wt.-% of bis-ethylhexyloxyphenol methoxyphenol triazine, at least 2 wt.-% of phenylbenzimidazol sulfonic in a cosmetically acceptable carrier and wherein the topical composition further comprises at least one further UVB and/or broadband filter substance.

9. Topical composition according to claim 7, wherein the topical composition further contains at least 3.6 wt.-% of octocrylene.

10. Topical composition according to claim 7, wherein the further UVB and/or broadband filter substance is selected from titanium dioxide, ethyl hexylsalicylate and/or homosalate.

11. Topical composition according to claim 7, wherein the total amount of the further UVB and/or broadband filter substance is in the range of 8 to 20 wt.-% with respect to the total weight of the topical composition.

12. Topical composition according to claim 7, further comprising at least 1 wt.-% of cetearyl alcohol (a mixture of C16 and C18 alcohol), Cetyl Alcohol and/or Glyceryl Myristate.
